(19) United States
(12) Patent Application Publication
(10) Pub. No.: US 2025/0370148 A1
(43) Pub. Date: Dec. 4, 2025

(54) POSITRONIUM LIFETIME IMAGE RECONSTRUCTION

(71) Applicant: The Regents of the University of California, Oakland, CA (US)

(72) Inventors: Jinyi Qi, Davis, CA (US); Bangyan Huang, Davis, CA (US)

(73) Assignee: The Regents of the University of California, Oakland, CA (US)

(21) Appl. No.: 18/873,599

(22) PCT Filed: Jul. 6, 2023
(86) PCT No.: PCT/US2023/069721

§ 371 Date: Dec. 10, 2024

Related U.S. Application Data
(60) Provisional application No. 63/358,787, filed on Jul. 6, 2022.
(60) Provisional application No. 63/511,955, filed on Jul. 5, 2023.

(51) Int. Cl. G01T 1/29

(52) U.S. Cl. CPC G01T 1/2985

(57) ABSTRACT

A system and methods are provided for constructing positron lifetime images based on the lifetimes of positronium (and/or positrons) emitted from a scanning subject into which a suitable radioactive tracer has been introduced. The system includes a positron emission tomography (PET) scanner having time of flight (TOE) capability that captures single events that may reflect any or all of a prompt gamma, a 511 keV annihilation photon, and a time delay between emission (or detection) of a prompt gamma and emission (or detection) of corresponding annihilation photons. Correlated singles are combined to form coincidence events indexed by the lines of response (LOR) in which they were detected. The disclosed methods use the coincidence events to generate images that reflect the lifetimes of the positronium (or positrons) within the subject, which may be used to identify a hypoxic region.

### RELATED APPLICATION(S)

This application claims the benefit of U.S. Provisional Patent Application No. 63/358,787, filed 6 Jul. 2022 (docket number UC22-579-1PSP), and U.S. Provisional Patent Application No. 63/511,955, filed 5 Jul. 2023 (docket number UC22-579-2PSP). The contents of the preceding applications are incorporated by reference herein.

### GOVERNMENT LICENSE RIGHTS

This invention was made with U.S. Government support by the National Institutes of Health under grant number R21EB032101. The U.S. Government has certain rights in the invention.

### BACKGROUND

This disclosure relates to the fields of electrical engineering and digital imagery. More particularly, a system, apparatus and methods are provided for reconstructing high-resolution positronium lifetime images.

Positron emission tomography (PET) enables the visualization of molecular processes in vivo, with the use of a positron-emitting radiotracer. A positron produces a pair of annihilation photons that can be detected and used to reconstruct images of the distribution of a radiotracer within a subject, with spatial resolution of approximately 3 mm using a clinical whole-body PET scanner. However, current PET imaging solutions have ignored the lifetime history of positrons before their annihilation.

It has been shown that the lifetime of positronium, the metastable pairing of one electron and one positron produced during PET scanning, is sensitive to the microenvironment of the surrounding tissue being imaged (e.g., the ambient oxygen pressure), and can be valuable for cancer staging and treatment planning. About 40% of positrons emitted during a PET scan form positronium that will decompose into photons, including para-positronium (p-Ps) and ortho-positronium (o-Ps); of these two types, ortho-positronium is particularly affected by its environment. Unfortunately, there is no practical method for imaging positronium lifetimes at a spatial resolution matching that of PET, due to the lack of proper image reconstruction methods.

In particular, the standard approach to gauging positronium lifetime uses a pair of time-of-flight (TOF) detectors to measure time differences between prompt gammas and corresponding photon annihilation, but only for uniform materials where no spatial localization is needed. For distributed or heterogeneous sources, the only way to localize the source position along the line of response (LOR) between two detectors is using the TOF information. However, state-of-the-art TOF resolution of modern PET scanners is around 250 ps-500 ps, which translates to a spatial localization uncertainty of 37 mm-75 mm, which is an order of magnitude worse than the spatial resolution of normal PET images. Furthermore, because of the large spatial uncertainty, positronium having different lifetimes may be mixed, which makes the conventional lifetime estimation method based on exponential curve fitting invalid. Therefore, a new method to reconstruct lifetime images is needed to make positronium lifetime imaging (PLI) practical using existing TOF PET.

### SUMMARY

In some embodiments, systems, apparatuses, and methods are provided for statistically reconstructing positronium (or positron) lifetime imaging (PLI) for use with a positron emission tomography (PET) scanner, to produce images having resolutions better than can be obtained with existing time-of-flight (TOF) systems. In these embodiments, positronium lifetime measurements can be used to measure dissolved oxygen concentration, and are independent of the concentration of tracer that emits the positrons. Therefore, hypoxic regions of a human body or other subject can be identified noninvasively, and medical treatment (e.g., of cancer) can be designed accordingly.

More particularly, whereas existing PET scanners simply track pairs of 511 keV annihilation photons produced by a positron's collision with an electron, an advanced PET scanner employed in some embodiments also captures the life history of a positron that merges with an electron to form positronium (Ps), prior to its annihilation, thereby noninvasively capturing information about the surrounding tissue.

Because both types of positronium—para-positronium (p-Ps) and ortho-positronium (o-Ps)—decay into photons with known lifetimes, and because the lifetime of positronium (especially o-Ps) is affected by the availability of unpaired electrons in the surrounding material, the advanced PET scanner and methods described herein for performing PLI allow characteristics of the surrounding human tissue (e.g., hypoxic regions) to be identified with precision based on how the positronium is affected by that tissue. For example, the annihilation rate of positronium depends on the frequency of interaction between the positronium and neighboring molecules. Whereas existing methods of performing positronium lifetime image reconstruction would require a PET scanner that provides better than 50 ps (picosecond) TOF resolution, which is unrealistic, methods provided herein are compatible with existing PET scanners (e.g., those with TOF resolution in the range of 250-700 ps), and yield resolutions of less than approximately 4 mm.

In embodiments disclosed herein, methods are provided for reconstructing the average lifetime image of positrons and/or positronium involving direct annihilations, annihilations of p-Ps, and/or annihilations of o-Ps. Assuming the fraction of positronium (e.g., the ratio of o-Ps to p-Ps) is constant (e.g., 25%, 30%, 40%), the average lifetime of the positronium is linearly related to the lifetime of the o-Ps (and the p-Ps) and therefore carries the same information as the lifetime of the o-Ps (e.g., interactions with surrounding tissue). The lifetime of the o-Ps can be measured with the help of tracers such as ⁴⁴Sc and ²²Na (and/or others), which emit a prompt gamma immediately after a B⁺ decay, and by recording the amount of time that elapses between detection of the prompt gamma (which indicates emission of a positron) and detection of the corresponding annihilation photons.

In some embodiments, in order to generate an average lifetime image of a subject, first a total activity image and a lifetime-weighted activity image are constructed for each voxel. The average lifetime image can then be reconstructed for each voxel as the ratio between the lifetime-weighted activity image and the total activity image for that voxel. The total activity image can be a standard PET image showing distribution of the radiotracer in the subject. The lifetime-weighted image may be constructed by assigning to each list-mode event a weight equal to the delay time, so that events with longer delays carry more weight in the reconstruction process.

In some embodiments, positronium lifetime imaging leverages a PET image reconstruction algorithm to reconstruct a series of images using time-thresholded data, after which curve-fitting is applied to determine the lifetime for each voxel. More particularly, triple coincidence events (each of which comprises detection of a pair of annihilation photons and a corresponding prompt gamma) are sorted based on the delay between photon annihilation and the prompt gamma, after compensating for their travel time difference. Then, for each given time threshold Tᵢ, triple coincidence events occurring within a time window ending at time Tᵢ are reconstructed using the ordered subset expectation maximization (OSEM) algorithm to produce an intermediate image. Finally, from intermediate images corresponding to a series of time thresholds, we obtain time-series images that can be used to estimate the positronium lifetime image for each voxel (e.g., through curve-fitting).

In some embodiments, a statistically based image reconstruction of positronium lifetime is based on an estimation of a maximum likelihood (ML) or penalized maximum likelihood (PML). Each PLI event is represented by a line of response (LOR) index iₖ determined by detections of the annihilation of paired photons and a time delay τₖ T. between the annihilation and detection of prompt gamma emission. When all o-Ps exhibit a common lifetime 1/λ, the time delay τₖ for a given event follows an exponential distribution that can be calculated as described below. Further, because the detection of any given event is independent of other events, a likelihood function of N events is also described below and allows calculation of the maximum likelihood estimate of λ for a homogeneous sample.

In a heterogeneous sample in which activity distribution and positronium lifetime are spatially variant, we estimate the activity concentration image beforehand using PET coincidence data. Based on the activity concentration of the tracer in voxel j (xⱼ) and the decay constant of o-Ps in voxel j (λⱼ, distribution of decay time t in LOR iₖ follows a distribution described below, and yields the probability of an event that was detected in LOR iₖ as having originated from voxel j.

The reconstructed image may be obtained by finding the maximizer of the probability density function. Because directly maximizing the probability density function is difficult and requires intensive processing, instead an optimization transfer principle may be applied to derive a separable surrogate function that is easier to maximize, and thereby obtain an iterative algorithm to find the solution.

To reduce noise in a reconstructed lifetime image, regularization can be introduced by including a penalty function in the objective function, which results in a PML reconstruction. One example of a penalty function is a pairwise penalty that penalizes the difference between the lifetime values of adjacent voxels. Other forms of penalty functions, including some used in standard PET image reconstruction, can be applied.

### DESCRIPTION OF THE FIGURES

FIG. 1 is a block diagram depicting system for constructing positronium (or positron) lifetime images, in accordance with some embodiments.

FIG. 2 is a flow chart demonstrating the SIMPLE method of reconstructing a positronium lifetime image, according to some embodiments.

FIG. 3 is a flow chart demonstrating the SIMPLE-M method of reconstructing a positronium lifetime image, according to some embodiments.

FIG. 4A is a table listing the types of tri-coincidences that may be detected in different time windows, according to some embodiments.

FIG. 4B illustrates prompt and delayed time windows for estimating the number of different types of random events, according to some embodiments.

FIG. 5 is a flowchart demonstrating a method of performing the SPLIT technique of positronium lifetime image reconstruction, with random events correction, according to some embodiments.

### DETAILED DESCRIPTION

The following description is presented to enable any person skilled in the art to make and use the disclosed embodiments, and is provided in the context of one or more particular applications and their requirements. Various modifications to the disclosed embodiments will be readily apparent to those skilled in the art, and the general principles defined herein may be applied to other embodiments and applications without departing from the scope of those that are disclosed. Thus, the present invention or inventions are not intended to be limited to the embodiments shown, but rather are to be accorded the widest scope consistent with the disclosure.

In some embodiments, methods, apparatus, and systems are provided for

performing positronium lifetime imaging (PLI). In these embodiments, measured lifetimes may be independent of the concentration of the positron-emitting tracer and, therefore, can be obtained with standard positron emission tomography (PET) imaging (e.g., the 2-meter EXPLORER scanner at the University of California at Davis Medical Center). As a result, simultaneous PET/PLI imaging is possible for the first time, to allow examination both of radiotracer distribution and the surrounding microenvironment.

Because of the effect dissolved oxygen concentration (pO₂) has upon the lifetime of positronium, one illustrative benefit of accurately measuring positronium lifetimes is the ability to measure a subject's pO₂, in vivo, and thereby detect a hypoxic region within the subject, which is important in the treatment of cancer and other health concerns. More particularly, techniques are provided for reconstructing images based on positronium lifetimes regarding a subject (e.g., a human body) of heterogeneous composition, with spatial resolutions identical or similar to standard PET images (e.g., 3 to 4 mm). In different embodiments, different techniques or methods may be used to reconstruct the positronium lifetime images from the available data.

About 40% or more of positrons emitted from radiotracers in tissue form positronium (Ps) with an electron before the paired entity annihilates, while others directly annihilate by colliding with an electron. In either event, the annihilation produces two 511 keV photons.

Positronium has two states that differ according to spin property. One is known as para-positronium (p-Ps), with the positron and electron having antiparallel spins, and the other is called ortho-positronium (o-Ps), with the positron and electron having parallel spins. Within human tissue, p-Ps has a lifetime of approximately 0.125 ns, while o-Ps has a lifetime of approximately 1.5-3 ns. When directly annihilated with an electron, a positron has a lifetime of about 0.4 ns. Inside tissue, the lifetime of o-Ps is reduced substantially by two types of interactions with surrounding molecules: pick-off annihilations and spin-exchange interactions. A pick-off annihilation occurs when the positron of a Ps annihilates with a foreign electron in a molecule. Spin-exchange interaction, which converts o-Ps to p-Ps, occurs when the surrounding molecules possess unpaired electrons. Both interactions reduce the lifetime of o-Ps and produce two 511 keV photons.

The positronium yield in water has been found to be 38% and the yield is expected to be higher in tissue. The ratio between o-Ps and p-Ps has been measured at approximately 3:1. Therefore, we can expect that about 30% or more of emitted positrons forms o-Ps that can be used for lifetime imaging measurements.

Because spin exchange interactions occur around two orders of magnitude more often than pick-off annihilations, the lifetime of o-Ps is sensitive to the concentration of paramagnetic molecules such as O₂. A technique based on positronium lifetime, called Positron Annihilation Lifetime Spectroscopy (PALS), has been developed in materials science to detect defects in metals, free volumes in polymers, and pores in porous materials. However, these measurements are conducted on uniform materials and, unlike human and animal imaging, do not require spatial localization. Using PALS, researchers have measured o-Ps lifetimes in normal and diseased tissues and found strong correlations between data obtained from PALS and histopathological examinations of the same tissue fragments.

Separately, the lifetime of o-Ps in water samples with different O₂ concentrations was studied. It was found that its lifetime decreases linearly with increasing pO₂, and the ability to distinguish hypoxic regions from control regions using o-Ps lifetimes was considered. Because hypoxic tumors are often resistant to radiation therapy and chemotherapy, the ability to identify hypoxic regions noninvasively in vivo will hopefully help develop more effective treatments.

To measure the lifetime of positronium, and o-Ps in particular, start and stop signals for making time measurements are needed. A useful start signal can be obtained when a positron emitter (i.e., a tracer) is employed that generates a prompt gamma when a positron is emitted. Illustrative radioisotopes that provide this signal and that have suitable half-lives include ⁴⁴Sc, ⁴⁷Sc, ⁶⁸Ga, ⁷⁶Br, and ⁸²Rb. As for a stop signal, detection of two 511 keV photons indicates that an o-Ps formed from the emitted positron has decomposed, and the length of time between the two events can be used to help estimate the lifetime of the o-Ps. This time-of-flight (TOF) information allows localization of a source along a given line of response (LOR) between two detectors.

It may be noted that image reconstruction for PLI is more complicated than for PET, due to the need to estimate both spatial location and positron lifetimes. To achieve a resolution comparable to PET images directly, a TOF resolution of about 50 ps would be required, which is unrealistic with present technology. Also, because o-Ps having different lifetimes may be mixed, the conventional lifetime estimation method based on exponential curve-fitting cannot be used.

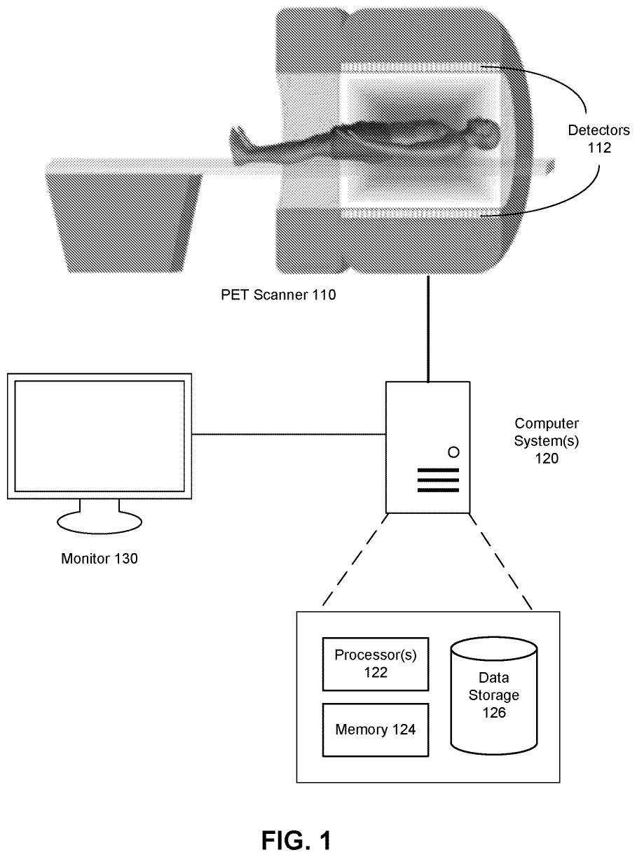

FIG. 1 is a block diagram of a system for constructing or reconstructing positronium (or positron) lifetime images, according to some embodiments.

System 100 comprises PET scanner 110, which is capable of capturing single events (i.e., individual detections of annihilation photons and of prompt gammas), and has a TOF resolution of about 250-700 ps. In some embodiments, PET scanner 110 is the total body EXPLORER PET scanner located at the medical center at the University of California at Davis, CA. System 100 also includes computer system 120 for performing computations described herein and monitor 130 for displaying partial and/or full positronium (or positron) lifetime images. Either or both computer system 120 and monitor 130 may be integral to scanner 110.

Detectors 112 within scanner 110 operate as with normal PET scanning to detect photons originating from a subject of a scanning operation, after a radioactive tracer is introduced into the subject to emit positrons. The detectors capture not only the location of detection of a photon, but also time of flight.

Multiple techniques for performing the image reconstruction are presented in the following sections. Each technique for constructing positron or positronium lifetime images involves collection of multiple coincidence events, wherein each event comprises a LOR index i, which is defined by positions at which a pair of annihilation photons are detected, and a time delay t between (a) a prompt gamma associated with emission of the positron that yielded the annihilation photons and (b) detection of the corresponding annihilation photons. Thus, the k^{th} event comprises LOR index ix and time delay τₖ.

In some or all techniques, the timing of coincidence events is adjusted to compensate for the travel times of the two annihilation photons and the prompt gamma. For example, an annihilation point within a subject of a PET scan is inferred deterministically based on the difference in the detection times of each photon in a pair of annihilation photons and the distances the photons traveled. Time tags of each coincidence event may thus be shifted backward to mark the time of emission of the corresponding positron.

### SIMPLE Technique

In some embodiments, reconstruction of positron lifetime images involves averaging all lifetime components or events. This technique is referred to as the SIMPLE (Statistical IMage reconstruction of Positron (or Positronium) Lifetime via timE-weighting) technique in order to distinguish it from other techniques described below.

It may be recalled that the lifetime of p-Ps does not vary much within biological tissue and, advantageously, the SIMPLE method of lifetime image construction or reconstruction correlates positron lifetimes with o-Ps lifetimes. Because the reconstruction is tied to the lifetime of o-Ps, it is sensitive to the microenvironment of the positrons and positronium and, in addition, no curve-fitting is required because no lifetime model is needed. Further, minimal modification is required to the ordered subset expectation maximization (OSEM) algorithm used to process lifetime events. The computation time and effort required to construct a lifetime image with this process are comparable to the construction of two standard PET activity images. Monte Carlo simulations using GATE (Geant4 Application for Tomographic Emissions) demonstrated the validity and accuracy of the resulting image.

When the fraction of positronium (Ps fraction) produced by a tracer is constant, meaning that percentage of positrons emitted by the tracer that form positronium remains relatively consistent over time, the average lifetime τ̅ of the positronium is related to the lifetime of ortho-positronium. Specifically, with a ratio of ortho-positronium (o-Ps) to para-positronium (p-Ps) of 3:1,

$$\overline{τ} = [ ( 0.25 \times p - Ps lifetime ) + ( 0.75 \times o - Ps lifetime ) ] \times Ps fraction \qquad ( A 1 )$$

The lifetime of para-positronium, which may be denoted τₚPₛ, is 0.125 ns, as indicated above, and equation (A1) can be solved for the lifetime of ortho-positronium, denoted τₒ-Pₛ, as follows:

$${τ}_{o - Ps} = 4 / 3 \times ( \overline{τ} / Ps fraction ) - ( {τ}_{p - Ps} / 3 ) \qquad ( A2 )$$

In some implementations of the SIMPLE method of lifetime image construction or reconstruction, an average lifetime image is generated by first constructing a total activity image x and a lifetime-weighted activity image w, wherein w=τ̅·x and · denotes element-wise multiplication. The average lifetime image can then be obtained as the ratio between the lifetime-weighted activity image and the total activity image. For example, a conventional OSEM algorithm may be applied to yield a standard (total) activity image x, while a modified OSEM algorithm (described below) may be applied to yield lifetime-weighted activity image w, and the desired lifetime image is yield by the ratio w/x.

As indicated above, each positron (or positronium) lifetime image event is denoted by a LOR index iₖ and a time delay τₖ. Thus, the total time delay of all lifetime events received in LOR i can be represented as

$${z}_{i} = {\sum }_{k ∈ {K}_{t}} {τ}_{k} = {\sum }_{j} {\sum }_{k ∈ {K}_{tj}} {τ}_{k} \qquad ( A 3 )$$

In equation (A3), Kᵢ is the set of all indices of PLI events detected in LOR i, and Kᵢⱼ is the subset of indices of the PLI events detected in LOR i that originated in voxel j, such that

$${K}_{i} = {⋃}_{j = 1}^{N} {K}_{i j} ,$$

wherein N is the total number of voxels (in LOR i that originated in voxel (i LOR i). An expectation or estimation of zᵢ can be expressed as

$${\overline{z}}_{i} = {\sum }_{j} E [ {\sum }_{k ∈ {K}_{ij}} {τ}_{k} ] = {\sum }_{j} {\overline{y}}_{ij} {\overline{τ}}_{j} \qquad ( A 4 )$$

In equation (A4), τ̅ⱼ is the expected or estimated time delay of PLI events originating in voxel j, while y̅ᵢⱼ is the estimated number of PLI events to be detected in LOR i that originated in voxel j, and is related to total activity image x such that y̅ᵢⱼ=Hᵢⱼxⱼ, wherein H is the standard system matrix employed in PET activity image reconstruction and Hᵢⱼ (the (i,j)^{th} element of the standard PET system matrix) represents the probability of detecting a pair of annihilation photons in LOR i resulting from a positron annihilation in voxel j. The preceding allows the relationship between z̅ᵢ and x to be expressed as

$${\overline{z}}_{i} = {\sum }_{j} {H}_{ij} {x}_{j} {\overline{τ}}_{j} = {\sum }_{j} {H}_{ij} {w}_{j} \qquad ( A 5 )$$

Thus, equation (A5) links lifetime-weighted activity image w to the estimated zᵢ, through a forward projection model. Now, to estimate w, the following derived list-mode OSEM algorithm is applied, wherein S, represents the nth subset:

$${w}_{j}^{( n + 1 )} = \frac{{w}_{j}^{( n )}}{{\sum }_{i} {H}_{tj}} {\sum }_{k ∈ {S}_{n}} \frac{{H}_{{t}_{k} j} {τ}_{k}}{{\sum }_{m} {H}_{{i}_{k} m} {w}_{m}^{( n )}} \qquad ( A 6 )$$

After reconstructing w, total activity image x can be reconstructed using the standard list-mode OSEM algorithm. Subsequently, the reconstructed lifetime image is obtained by calculating the ratio wⱼ/xⱼ for each voxel j.

Performing positron lifetime image reconstruction using the SIMPLE method yields quantitatively accurate positron average lifetime images, with high spatial resolution, using an existing PET scanner having commercially available time-of-flight resolution. This method has relatively low computational burdens because it requires only one lifetime-weighted OSEM reconstruction (in addition to the conventional activity image reconstruction), thereby yielding a reconstructed image for the same effort as two standard PET activity images.

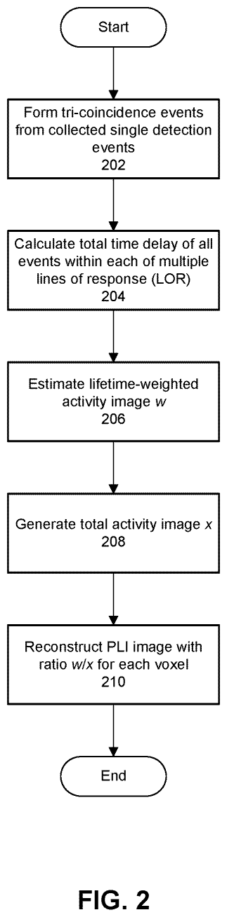

FIG. 2 is a flow chart demonstrating the SIMPLE method of reconstructing a positronium lifetime image, according to some embodiments.

In operation 202, a PET scanner scans a subject containing a radioactive tracer that emits positrons. The scanner detects and record single events associated with emission or annihilation of a positron, and/or of annihilation of a positronium formed from a positron. For example, the scanner (or an associated computer system) may calculate the time of flight (TOF) of each detected photon and the time delay between two 511 keV annihilation photons and a prompt gamma. These data are combined to form coincidence (or tri-coincidence) events, which are indexed by the line of response (LOR) within which the events were detected.

In operation 204, for each of multiple LORs, the total time delay of all events within the LOR is calculated, using equation (A4) above, for example.

In operation 206, a lifetime-weighted activity image (w) is then estimated using the standard PET system matrix and time delays calculated for events captured by the scanner. This may involve the application of equation (A6) above.

In operation 208, a total activity image (x) is generated from the collected events using a standard list-mode OSEM algorithm.

In operation 210, the constructed/reconstructed positronium lifetime image is then generated by calculating, for each voxel, the ratio of lifetime-weighted activity image w by total activity image x, and displaying the result.

### SIMPLE-M Technique

An improvement to the SIMPLE technique for positronium or positron lifetime image reconstruction, referred to herein as SIMPLE-M, has a similar computational burden (also necessitating the computational burden associated with reconstructing two or three standard PET activity images), without curve fitting, but (unlike SIMPLE) can allow direct estimation of the o-Ps lifetime through inclusion of higher orders of moments. In contrast, the SIMPLE method described above reconstructs just the first moment positronium lifetime image m, which is the average lifetime of all interaction pathways (i.e., as o-Ps, p-Ps, or direct annihilation).

In the absence of prior knowledge about lifetime, 2n-1 moment images are required to solve a lifetime model with n interaction pathways. For a typical three-pathway model, this means the first to the fifth moments. However, since the occurrence ratio of o-Ps to p-Ps is known, it is reasonable to reduce one moment image. To obtain a simple closed-form solution to equations (B3) and (B4), below, it is assumed that the lifetimes of p-Ps and direct annihilation are fixed and known in biological tissue.

A slightly high bias may be encountered with the SIMPLE-M method because the variance of the intensity-weighted moment image wⁿ increases as the moment order increases, leading to a more biased estimator. However, reconstruction of high-order moment images may be avoided not only to simplify the process of solving the o-Ps lifetime, but also to reduce bias in the reconstructed lifetime images.

As with SIMPLE, in the SIMPLE-M method, events are characterized by a corresponding line of response (LOR) iₖ, which is determined by the locations of detection of paired annihilation photons and the corresponding time delay between (a) emission of the prompt gamma associated with the positron that yielded the photon pair and (b) the annihilation of the positron. In the SIMPLE-M technique, distribution modeling of the time delays of events originating from voxel j may be represented as a summation of multiple exponential decays convolved by a Gaussian function g(τ) characterized by the time resolution of the PET scanner being used, as follows:

$$f ( τ \vert {A}_{l , j} , {λ}_{l , j} , l ∈ \{ o , p , d \} ) = g ( τ ) * {\sum }_{l ∈ \{ o , p , d \}} {A}_{l , j} {λ}_{l , j} exp ( - {λ}_{l , j} τ ) u ( τ ) \qquad ( B 1 )$$

As already indicated, three pathways of annihilation are recognized, including o-Ps, p-Ps, and direct annihilation; these pathways are denoted o, p, and d, respectively. In equation (B1), A_{i,j} is the probability that a positron or positronium will annihilate in voxel j via the indicated pathway. In other words, it may be considered the fraction or percentage of annihilations within voxel j that are determined or estimated to involve pathway l (wherein l is either o, p, or d), while λ_{i,j} is the decay rate (i.e., the inverse of lifetime).

Using the above model, lifetime estimations can be computed using moments, wherein the n^{th} moment of a lifetime is written as

$${m}_{j}^{n} = E [ {τ}^{n} ] = {\int }_{- ∞}^{∞} {τ}^{n} f ( τ \vert {A}_{l , j} , {λ}_{l , j} , l ∈ \{ o , p , d \} ) d τ \qquad ( B 2 )$$

Equation (B2) can be reduced to

$${m}_{j}^{n} = {\sum }_{s = 0}^{n} \frac{n !}{( n - s ) !} {μ}_{n - s} {G}_{s , j} \qquad ( B 3 )$$

Within equation (B3), μₖ=E_{g(τ)}[τ^{k}] and denotes the k^{th} moment of Gaussian distribution g(τ), while G_{s,j} are moment images up to a scaling factor of s! (s-factorial), and

$${G}_{s , j} = {\sum }_{l ∈ \{ o , p , d \}} \frac{{A}_{l , j}}{{λ}_{l , j}^{s}} \qquad ( B 4 )$$

From equations (B3) and (B4), lifetimes and intensities can be estimated by calculating an empirical estimate of moments mⱼⁿ, determining G_{s,j} from equation (B3), and then estimating the lifetimes and fractions of all annihilations via each pathway from equation (B4). Without prior information regarding their lifetimes and intensities, six moment equations (the zeroth through the fifth) are required for the six unknown values (Aₗ and λₗ, for l ⊂ {o, p, d}).

However, because the ratio of o-Ps to p-Ps is known to be 3:1, the ratio of Aₒ to Aₚ likewise is 3:1. Also, we may assume that the lifetimes of p-Ps and direct annihilations are 0.125 ns and 0.4 ns, respectively. Therefore, only three moment equations are needed (the zeroth through the second), and the closed-form solution of λₒ is found using the quadratic equation:

$${λ}_{o} = \frac{- B + \sqrt{{B}^{2} - 4 AC}}{2 A} \qquad ( B 5 )$$

In equation (B5), variables A, B, and C are as follows, wherein voxel j is omitted in the interest of brevity:

$$A = {G}_{0} {λ}_{a} - {G}_{1} {λ}_{a}^{2} - {G}_{0} {λ}_{p} + {G}_{2} {λ}_{p} {λ}_{a}^{2} + 4 {G}_{1} {λ}_{p}^{2} - 4 {G}_{2} {λ}_{p}^{2} {λ}_{a}$$

$$B = - 3 {G}_{0} {λ}_{p}^{2} + 3 {G}_{2} {λ}_{p}^{2} {λ}_{d}^{2}$$

$$C = 3 {G}_{0} {λ}_{p}^{2} {λ}_{a} - 3 {G}_{1} {λ}_{p}^{2} {λ}_{a}^{2}$$

Estimation of positronium lifetime images using the SIMPLE-M method requires estimation of moment images from measured lifetime events, which is done by constructing intensity-weighted moment image

$${w}_{j}^{n} = {x}_{j} {m}_{j}^{n}$$

and dividing it by activity image x, which is constructed using the paired annihilation photons of the lifetime events. Construction of the intensity-weighted moment image wⁿ requires a projection that matches the expectation or estimate of the forward projection of wⁿ:

$${z}_{i}^{\overline{n}} = {\sum }_{j} {H}_{ij} {w}_{j}^{n} = {\sum }_{j} {H}_{ij} {x}_{j} {m}_{j}^{n} = {\sum }_{j} E [ {y}_{ij} ] {m}_{j}^{n} \qquad ( B 6 )$$

In equation (B6), H is the standard PET system matrix, and yᵢⱼ denotes the number of events that originated in voxel j and were detected in LOR i. An empirical estimate of

$${m}_{j}^{n}$$

is written as

$$= \frac{1}{{y}_{ij}} {\sum }_{k ∈ {K}_{i j}} {τ}_{k}^{n} \qquad ( B 7 )$$

In equation (B7), Kᵢⱼ is the set of list-mode events that originated in voxel j and were detected in LOR i, and

$${K}_{i} = {⋃}_{j = 1}^{N} {K}_{ij} .$$

Therefore, we obtain

$${\sum }_{j} {H}_{ij} {w}_{j}^{n} = {\sum }_{j} E [ {\sum }_{k ∈ {K}_{ij}} {τ}_{k}^{n} ] = E [ {\sum }_{k ∈ {K}_{i}} {τ}_{k}^{n} ] \qquad ( B8 )$$

Equation (B8) shows that the intensity-weighted moment image can be estimated from time-delay-weighted projection data:

$${z}_{i}^{n} = {\sum }_{k ∈ {K}_{i}} {τ}_{k}^{n} \qquad ( B9 )$$

To reconstruct intensity-weighted moment image wⁿ, in the SIMPLE-M technique a list-mode OSEM algorithm is applied with the following updating equation:

$${w}_{j}^{{n}^{( r + 1 )}} = \frac{{w}_{j}^{{n}^{( r )}}}{{\sum }_{i} {H}_{ij}} {\sum }_{k ∈ {S}_{r}} \frac{{H}_{{i}_{k} j} {τ}_{{i}_{k}}^{n}}{{\sum }_{i} {H}_{{i}_{k} j} {w}_{j}^{{n}^{( r )}}} \qquad ( B10 )$$

In equation (B10), Sᵣ is the r^{th} subset of the list-mode event indices. Moment image mⁿ can now be obtained by taking the voxel-wise ratio of image w^{th} over activity image x.

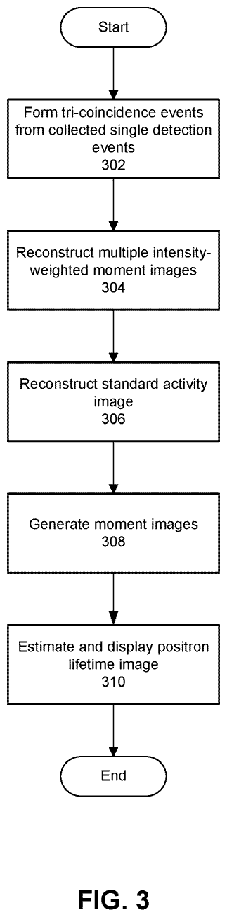

FIG. 3 is a flow chart demonstrating the SIMPLE-M method of reconstructing a positronium lifetime image, according to some embodiments.

In operation 302, a PET scanner scans a subject containing a radioactive tracer that emits positrons. The scanner detects and record single events associated with emission or annihilation of a positron, and/or of annihilation of a positronium formed from a positron. For example, the scanner (or an associated computer system) may calculate the time of flight (TOF) of each detected photon and the time delay between two 511 keV annihilation photons and a prompt gamma. These data are combined to form coincidence (or tri-coincidence) events, which are indexed by the line of response (LOR) within which the events were detected.

In operation 304, a series of intensity-weighted moment images

$${w}_{j}^{n}$$

is reconstructed. In some embodiments, equation (B10) above may be used for this reconstruction.

In operation 306, a standard activity image x is reconstructed using the standard OSEM algorithm and 511 keV coincidence events in the tri-coincidence data, and the moment images

are obtained by taking the ratio between

$${w}_{j}^{n}$$

and x. In particular,

$$= \frac{{w}_{j}^{n}}{{x}_{j}} .$$

In operation 308, values of G_{s,j} are determined by solving the set of linear equations (B3) above.

Finally, in operation 310, the o-Ps lifetime image is estimated using equation (B5) above, after which it is displayed (e.g., on one or more monitors coupled to the PET scanner). Because one or more techniques described herein generate three-dimensional lifetime information, the display of the final (estimated) lifetime image can provide three-dimensional views.

### SPLIT Technique

In some other embodiments, an image reconstruction technique termed SPLIT (Statistical Positronium (or Positron) Lifetime Image reconstruction via time-Thresholding) involves sorting lifetime events according to multiple thresholds, generating lifetime-encoded activity images for each threshold (e.g., using ordered subset expectation maximization (OSEM)), and then fitting the images to a curve using a lifetime model to find the lifetime associated with each voxel. The computational cost is higher than with SIMPLE and SIMPLE-M, but the method leverages existing reconstruction algorithms to produce a threshold-activity curve for each voxel and then estimates the lifetime image from the curves.

As with SIMPLE-M, a Gaussian function is used to model the distribution of time delays associated with coincidence events:

$$p ( τ \vert λ , A ) = g ( τ ) * {\sum }_{l ∈ \{ o , p , d \}} {A}_{l} {λ}_{l} exp ( - {λ}_{l} τ ) u ( τ ) \qquad ( C1 )$$

In equation (C1), g(τ) is a Gaussian fraction with a FWHM (Full Width at Half Maximum) equal to

$$\frac{\sqrt{3}}{2}$$

times the FWHM of the coincidence revolving time (CRT) of the PET scanner being used, because the time delay is the period of time between a prompt gamma associated with a positron emission and the (average of the times of) detection of the resulting two 511 keV annihilation photons. Thus, τ=[(t₁+t₂)/2]−τ_{pg}, wherein t₁ and t₂ are the times of detection of the annihilation photons and τ_{pg} is the time of detection of the prompt gamma.

Also in equation (C1), u(t) is the unit step function (i.e., u(t)=1 when t>0 and u(t)=0 when t<0), and/represents a positron/positronium interaction pathway as described above, wherein pathways o, p, and d correspond to o-Ps, p-Ps, and direct annihilation, respectively. Aₜ denotes the fraction of an interaction pathway and λₗ is the decay rate (i.e., the inverse of lifetime). Because the lifetimes of para positronium and positrons that directly annihilate upon interaction with an electron are short and relatively fixed in duration, λₒ is the primary lifetime of interest in techniques described herein, and λ and A are used to refer to unknown lifetimes and fractions in the various mathematical equations and expressions that are presented.

One characteristic of the SPLIT technique is that it involves decoupling lifetime estimation and image reconstruction by obtaining a lifetime-encoded activity image zⱼ(T_{c}) for each voxel j, wherein T_{c} is a time threshold and only lifetime events having time delays less than the threshold are included in the image. Because lifetime events are independent of each other, and the total number of events originating from a given voxel j is Poisson-distributed, the estimated or probable number of retained events (those having lifetime less than T_{c}) can be represented as P(T_{c}; λⱼ,Aⱼ)×xⱼ, wherein xⱼ is the activity image for voxel j that can be estimated from all lifetime events. Based on this, a forward projection model that links (a) the expected number of lifetime events that meet the threshold requirement and (b) the activity image zⱼ(T_{c}) can be written as

$${\overline{g}}_{i} = {\sum }_{j} {H}_{ij} [ {\int }_{- ∞}^{{T}_{c}} p ( τ \vert {λ}_{j} , {A}_{j} ) d τ ] {x}_{j} = {\sum }_{j} {H}_{ij} P ( {T}_{c} ; {λ}_{j} , {A}_{j} ) {x}_{j} = {\sum }_{j} {H}_{ij} {z}_{j} ( {T}_{c} ) \qquad ( C2 )$$

In equation (C2), g̅ᵢ is the expected number of retained events in LOR i, and Hᵢⱼ is the (i,j)^{th} element of the standard PET matrix, which indicates the probability of detecting a pair of annihilation photons in LOR i from a corresponding positron annihilation in voxel j.

Next, lifetime-encoded images are reconstructed using a set of thresholds

$$\{ {T}_{c}^{m} \vert m = 1 , … , M \} .$$

For each threshold

$${T}_{c}^{m} ,$$

events with time delays less than the threshold are reconstructed according to the ordered subset expectation maximization (OSEM) algorithm, which generates estimate \hat{z}^{m} of the lifetime-encoded activity concentration:

$${z}_{j}^{m} = {x}_{j} P ( {T}_{c}^{m} ; {λ}_{j} , {A}_{j} ) , m = 1 , … , M \qquad ( C3 )$$

Then, the o-Ps lifetime in each voxel j is obtained by minimizing the squared error:

$${λ}_{j} , {A}_{j} = {arg min}_{{λ}_{j} , {A}_{j}} {\sum }_{m = 1}^{M} {[ {z}_{j}^{m} - {x}_{j} P ( {T}_{c}^{m} ; {λ}_{j} , {A}_{j} ) ]}^{2} \qquad ( C4 )$$

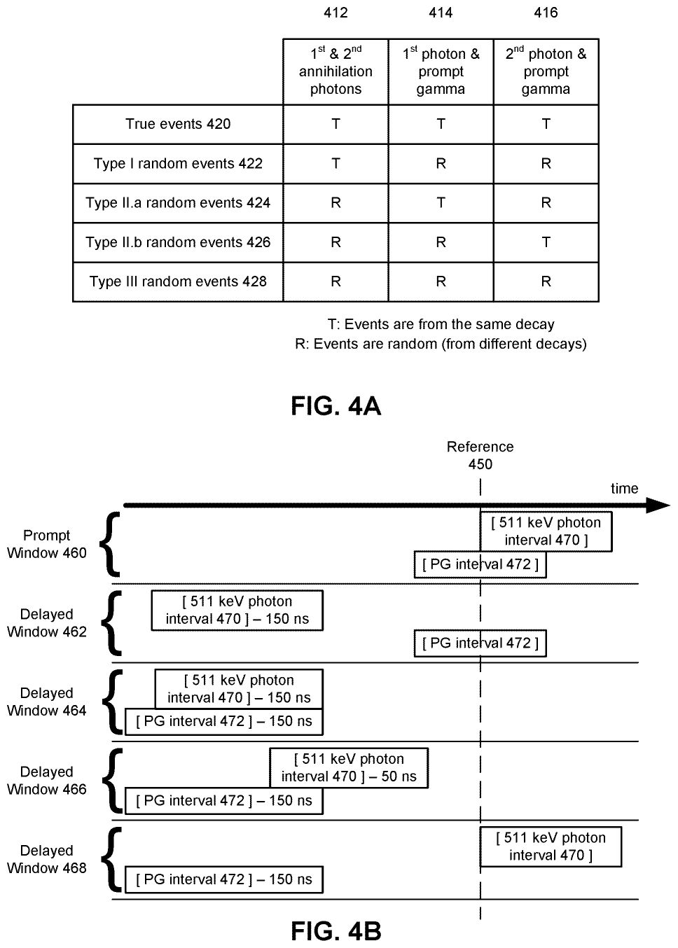

In embodiments of the SPLIT technique described herein, individual detections (i.e., of prompt gamma emissions, of 511 keV photons) are grouped to form coincidence events, or tri-coincidence events since each event involves three detections. During the grouping process, a list of single events is traversed, and every annihilation photon detection is used as a reference in search of a matching prompt gamma and a matching annihilation photon detection, within their respective time windows. However, some tri-coincident events may be formed by grouping random or uncorrelated detection events. Therefore, random event correction may therefore be necessary in order to obtain a more accurate final lifetime image, and may be conducted as described below with reference to FIGS. 4A-B.

After random event correction is performed, the SPLIT process for constructing or reconstructing a PLI continues as described here. The event correction process described below yields a truncated probability density function for time delays as follows, wherein Fis a scaling factor for normalizing the total probability to 1, it is assumed that the support of time delays is [T₁,T₂], and B is the constant background caused by random events:

$$p ( τ \vert λ , A ) = \{ \begin{matrix}\frac{1}{F} [ g ( τ ) ⋆ {\sum }_{l = o , p , d} {A}_{l} {λ}_{l} exp ( − {λ}_{l} τ ) u ( τ ) + B ] , τ ∈ [ {T}_{1} , {T}_{2} ] \\ 0 , otherwise\end{matrix} \qquad ( C 5 )$$

Now, equation (C3) can be rewritten by substituting (C5) and regrouping some variables, so that

$${x}_{j} ( {T}_{1} , {T}_{c}^{m} ) = {x}_{j} ( {T}_{1} , {T}_{2} ) {\int }_{{T}_{1}}^{{T}_{c}^{m}} p ( τ \vert {λ}_{j} , {A}_{j} ) d τ = {G}_{j} {\int }_{{T}_{1}}^{{T}_{c}^{m}} ( g ( τ ) * {\sum }_{l = o , p , d} {A}_{l , j} {λ}_{l , j} exp ( - {λ}_{l , j} τ ) u ( τ ) + {B}_{j} ) d τ \qquad ( C 6 )$$

In equation (C6), xⱼ(T₁, T₂) is the lifetime-encoded image for voxel j estimated by all events within time interval [T₁, T₂], and zⱼ(T_{c})=xⱼ(−∞, T_{c}). Activity xⱼ(T₁, T₂) and the normalization factor Fⱼ reduce to

$${G}_{j} = \frac{{x}_{j}}{{F}_{j}} ,$$

which is a constant as

$${x}_{j} ( {T}_{1} , {T}_{2} ) ∝ {\int }_{{T}_{1}}^{{T}_{2}} p ( τ \vert {λ}_{j} , {A}_{j} ) d τ .$$

The threshold activity curve is evaluated in both a linear range (e.g., 50-100ns), for estimating the quantity of type I random events, and a non-linear range (e.g., 0-20 ns) for estimating the quantity of type II and type III events. Separate estimations reduce the complexity of curve-fitting in the non-linear range. Thus, in the linear range, and because the integral of the exponential functions converges to 1 when

$${T}_{c}^{m} ∈ [ 50 , 100 ]$$

ns, equation (C6) can be approximated as

$${x}_{j} ( {T}_{1} , {T}_{c}^{m} ) = {G}_{j} {B}_{j} {T}_{c}^{m} + {G}_{j} ( 1 − {B}_{j} {T}_{1} ) \qquad ( C 7 )$$

Fitting this linear curve of

$${x}_{j} ( {T}_{c}^{m} )$$

yields slope GⱼBⱼ and the intercept Gⱼ(1−BⱼT₁), thereby obtaining Gⱼ and Bⱼ. We then solve for the parameters of interest by minimizing the mean squared error between the reconstructed lifetime-encoded activity curve and their expected values on a voxel-by-voxel basis. Specifically, λⱼ, Aⱼ=

$${arg min}_{{{λ}_{j} {A}_{j}}_{}} {{\sum }_{m - 1}^{M} [ {\hat{x}}_{j} ( {T}_{1} , {T}_{c}^{m} ) - {G}_{j} {\int }_{{T}_{1}}^{{T}_{c}^{m}} ( g ( τ ) * {\sum }_{l - o , ρ , d} {A}_{l , j} {λ}_{l , j} exp ( - {λ}_{l , j} τ ) u ( τ ) + {B}_{j} ) d τ ]}^{2} \qquad ( C 8 )$$

In equation (C8),

$${\hat{x}}_{j} ( {T}_{1} , {T}_{c}^{m} )$$

denotes the reconstructed lifetime-encoded images, and M is the number of time thresholds in the non-linear range.

Event correction is now described, during which random coincidences are estimated (e.g., in each LOR). To do so, each photon-detection event (i.e., detection of a 511 keV annihilation photon) is used as a reference for finding the other photon that originated from the same annihilation and also for finding the prompt gamma emission corresponding to emission of the positron that decayed into the pair of 511 keV photons, within their respective time windows. The combination of these three events/items is one coincidence (or tri-coincidence) event. It should be noted that correction for random events (e.g., detections of random or uncorrelated annihilation photons or prompt gammas) is more complex than in standard PET because there are multiple types or categories of random tri-coincidences, as illustrated in FIGS. 4A-B. It may also be noted that random event correction and/or travel distance correction may be applied in any of the various image reconstruction techniques described herein.

FIG. 4A is a table listing the types of tri-coincidences that may be detected in different time windows, according to some embodiments.

Five types of events are listed, including true events 420, wherein all three components of a true coincidence event are properly combined (i.e., a prompt gamma and both resulting 511 keV annihilation photons), and three types (Types I, II, and III) of random events 422-428 in which at most two detections are from the same decay (i.e., an annihilation photon is matched with either (but not both) its paired photon or its prompt gamma). Random events 422-428 have different characteristics depending on which (if any) events from the same decay are grouped.

In FIG. 4A, column 412 indicates whether each type of events involves a detected annihilation photon correctly matched with its mate from the same positron annihilation, column 414 indicates whether the detected annihilation photon is combined with the corresponding prompt gamma, and column 416 indicates whether the detected annihilation photon is the later photon from a pair and was matched with the correct prompt gamma. Specifically, ‘T’ indicates that the two indicated events are from the same positron decay, while ‘R’ indicates that the two events are from different (e.g., random) decays.

The random events or outcomes are denoted as Types I, II.a, II.b, and III because there are three types of random tri-coincidences in a prompt gamma time window, depending on the relationship among the three single events as shown in FIG. 4A. Type I random events 422 involve a pair of 511 keV photons from the same annihilation but with a random prompt gamma (i.e., a prompt gamma associated with a positron other than the one that decayed into the paired photons); Type II events 424, 426 involve a 511 keV photon and the prompt gamma from the same annihilation, but with a random 511 keV photon in place of the matching photon; Type III events 426 involve detections of photons from three different annihilations. Type II and type III random events must be corrected for during reconstruction of lifetime-encoded activity images, as background events in the projection domain, because they consist of two unrelated 511 keV photon detections, similar to random coincidences in conventional PET imaging. Type I random events may be corrected either during image reconstruction or during the lifetime curve fitting as a constant background after the reconstruction.

For each reference event (511 keV annihilation photon detection), one prompt time window and three delayed time windows are defined, as shown in FIG. 4B. Tri-coincidences formed within the prompt time window are referred to as prompt events, and tri-coincidences formed within a delayed time window are referred to as delayed events.

FIG. 4B illustrates prompt and delayed time windows for estimating the number of different types of random events, according to some embodiments. In FIG. 4B, reference 450 indicates a time associated with a 511 keV annihilation photon (e.g., the detection time or the emission time). Annihilation (511 keV) photon time interval 470 is defined as the time interval [0,T₅₁₁], which represents the maximum time difference between two 511 keV photons, based on the difference in their travel distances. Specifically, T₅₁₁ is the upper-bound of time that may lapse between a first annihilation photon detection (i.e., reference 450) and detection of the paired photon, and is usually between 2 ns and 7 ns. Prompt gamma (or PG) time interval is the time interval

$$[ - {T}_{c}^{m} , {T}_{1} ] , wherein {T}_{c}^{m}$$

and T₁ is the lower bound of the time delay, as used in equation (C6).

Prompt window 460 reflects alignment of a prompt-gamma time window 472 with an annihilation photon time window 470 for true tri-coincidence events, but may also or instead encompass a random event of any of the three types. In each of delayed windows 462 and 464, one 511 keV photon detection is unaligned or uncorrelated with the other two detections (the prompt gamma and the other annihilation photon), as evidenced by the long time delay. In delayed window 466, all three detections are random and uncorrelated with each other. Delayed windows 462-466 are used, respectively, to estimate the quantity of type II.a and type III events, type II.b and type III events, and type III events. Delayed window 468 is used to evaluate type I random events.

Using the results of delayed windows 462-466, an additive factor denoted

$${r}_{i}^{m}$$

is estimated as follows, wherein [delayed window

$${\# k ]}_{i}^{m}$$

is the number of lifetime events that occurred in delayed window k, in LOR i, with time delay less than

$${T}_{c}^{m} :$$

$${r}_{i}^{m} = {⌈ delayed window 462 ⌉}_{i}^{m} + {⌈ delayed window 464 ⌉}_{i}^{m} - {⌈ delayed window 466 ⌉}_{i}^{m}$$

The additive factor is used to modify equation the forward projection model of (C2) for random correction, as follows:

$${\overline{g}}_{i}^{m} = {\sum }_{j} {H}_{i j} {z}_{j}^{m} + {r}_{i}^{m}$$

This random events correction is applied to every lifetime-encoded image, and therefore the width of the time interval for prompt gammas is determined by the time threshold.

Type I random events are corrected in the fitting of the threshold-activity curve because each type I random event consists of a true 511-keV coincidence and a random prompt gamma, which means its time delay is uniformly distributed throughout its support. It is convenient to reconstruct type I random events and true lifetime events as a whole, and then use a flat background to model the random events in the time delay distribution. Alternatively, one could reconstruct a first image using only type I random events and reconstruct a second image using both type I random events and true lifetime events, and then subtract the first image from the second. Following event correction, image reconstruction can proceed as described above.

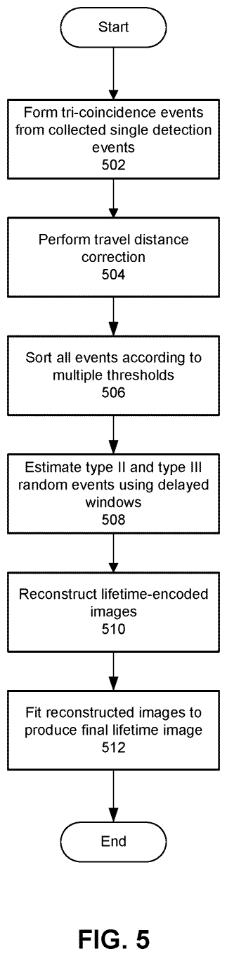

FIG. 5 is a flowchart demonstrating a method of performing the SPLIT technique of positronium lifetime image reconstruction, with random events correction, according to some embodiments.

In operation 502, single events are grouped to form tri-coincidence groupings. A single event involves detection of a prompt gamma or an annihilation photon (which may be either of a paired set of 511 keV photons produced by annihilation of a positron). A given tri-coincidence group is formed based on detection times (according to prompt window 460 of FIG. 4B), and may or may not be valid, meaning that the three events reflected in the group may or may not be associated with or have resulted from the same positron.

In operation 504, travel distance correction is applied to compensate for the different travel times of a prompt gamma and of each detected annihilation photon. Based on this correction, the timestamp or time tag of a particular tri-coincident event is shifted backward to the estimated time of emission of the corresponding positron. Operation 504 yields a collection of tri-coincidences that include prompt and delayed events.

In operation 506, time delay thresholds are applied to divide the tri-coincidence events into multiple buckets or intervals. In particular, multiple time delay thresholds are identified, and the events are sorted into them. Equation (C2) above may be used to estimate the number of retained events, in each LOR, for each voxel. In some embodiments, thresholds are uniformly distributed in time, such as every 0.2 ns from 0 to 20 ns. In other embodiments, thresholds are non-uniformly distributed over the same period of time (20 ns) or some other time period.

In operation 508, an estimate of type II and type III random events is obtained using delayed windows 462-468 of FIG. 4B. This operation yields, for each of multiple thresholds, sets of list-mode events and random additive factors for each threshold applied in operation 506.

In operation 510, corrected lifetime-encoded images are reconstructed using an OSEM algorithm. In some embodiments, the reconstruction field of view may be on the order of 50 mm×50 mm×180 mm, with a voxel size of approximately 0.8 mm×0.8 mm×1.6 mm, which is equivalent to half the pitch of a detector crystal.

In operation 512, corrected reconstructed lifetime-images are fitted to threshold-activity curves, which may be done using the MATLAB built-in function fmincon for each voxel, to find the o-Ps lifetime and intensity of each interaction pathway and yield the final positronium lifetime image. The lifetimes of p-Ps and direct annihilations may be fixed at 0.125 ns and 0.4 ns, respectively. After operation 512, the method ends.

An environment in which one or more embodiments described above are executed may incorporate a general-purpose computer or a special-purpose device such as a hand-held computer or communication device. Some details of such devices (e.g., processor, memory, data storage, display) may be omitted for the sake of clarity. A component such as a processor or memory to which one or more tasks or functions are attributed may be a general component temporarily configured to perform the specified task or function, or may be a specific component manufactured to perform the task or function. The term “processor” as used herein refers to one or more electronic circuits, devices, chips, processing cores and/or other components configured to process data and/or computer program code.

Data structures and program code described in this detailed description are typically stored on a non-transitory computer-readable storage medium, which may be any device or medium that can store code and/or data for use by a computer system. Non-transitory computer-readable storage media include, but are not limited to, volatile memory; non-volatile memory; electrical, magnetic, and optical storage devices such as disk drives, magnetic tape, CDs (compact discs) and DVDs (digital versatile discs or digital video discs), solid-state drives, and/or other non-transitory computer-readable media now known or later developed.

Methods and processes described in the detailed description can be embodied as code and/or data, which may be stored in a non-transitory computer-readable storage medium as described above. When a processor or computer system reads and executes the code and manipulates the data stored on the medium, the processor or computer system performs the methods and processes embodied as code and data structures and stored within the medium.

Furthermore, the methods and processes may be programmed into hardware modules such as, but not limited to, application-specific integrated circuit (ASIC) chips, field-programmable gate arrays (FPGAs), and other programmable-logic devices now known or hereafter developed. When such a hardware module is activated, it performs the methods and processes included within the module.

The foregoing embodiments have been presented for purposes of illustration and description only. They are not intended to be exhaustive or to limit this disclosure to the forms disclosed. Accordingly, many modifications and variations will be apparent to practitioners skilled in the art. The scope is defined by the appended claims, not the preceding disclosure.

What is claimed is:

1. A method of generating a positron lifetime image, comprising:
operating a Positron Emission Tomography (PET) scanner to collect coincidence events involving a subject of the scanning, wherein each coincidence event involves (a) a line of response (LOR) in which photons caused by annihilation of a positron are detected and (b) a time delay between a prompt gamma associated with emission of the positron and the detection of the photons;
for each of multiple voxels of the subject of the PET scanning, estimating a spatial distribution of the annihilated positrons and lifetimes of the positrons or of positronia formed from the positrons; and
generating and displaying the positron lifetime image of the subject.

2. The method of claim 1, further comprising:
constructing a total activity image based on the detected photons;
constructing one or more estimated intensity-weighted moment images based on the quantity of coincidence events detected in each of multiple LORs; and
generating the positron lifetime image by determining, for each of the multiple voxels, the ratio of the intensity-weighted moment images over the activity image and calculating the lifetime image from the moment images.

3. The method of claim 2, wherein:
generating the activity image comprises, for each coincidence event, correlating the detected photons to a voxel from which the photons originated.

4. The method of claim 2, further comprising:
modeling the time delays of coincidence events as a summation of exponential decays convolved by a Gaussian function characterized by a time resolution of the PET scanner.

5. The method of claim 1, further comprising:
constructing a total activity image based on the detected photons;
constructing a lifetime-weighted image based on the coincidence events detected in each of multiple LORs; and
constructing the positron lifetime image by determining, for each of the multiple voxels, the ratio of the lifetime-weighted image to the total activity image.

6. The method of claim 5, further comprising:
aggregating the time delays of coincidence events in each of multiple lines of response (LOR);
weighting the coincidence events by their time delays; and
summing the weights of the coincidence events.

7. The method of claim 1, further comprising:
sorting the coincidence events into multiple time intervals based on the time delays;
for each of the multiple time intervals, using the coincidence events occurring during the window to generate an intermediate image associated with the time window; and
fitting a curve to the multiple intermediate images to produce, for each of multiple voxels within the subject, a corresponding portion of the positron lifetime image.

8. The method of claim 7, further comprising:
iterating between updating the intermediate image using the estimated lifetimes and fitting a curve to the intermediate images to obtain the positron lifetime image.

9. The method of claim 7, wherein each coincidence event further comprises:
detection times of two annihilation photons caused by annihilation one or more positrons;
and a detection time of a prompt gamma.

10. The method of claim 1, further comprising:
performing travel distance correction to adjust the timestamps of one or more of the coincidence events.

11. The method of claim 1, further comprising:
performing random events correction to correct for one or more coincidence events that include detection times associated with annihilation of more than one positron.

12. A system for generating a positron lifetime image, the system comprising:
one or more processors; and
memory for storing instructions that, when executed, cause the one or more processors to:
operate a Positron Emission Tomography (PET) scanner to collect coincidence events involving a subject of the scanning, wherein each coincidence event involves (a) a line of response (LOR) in which photons caused by annihilation of a positron are detected and (b) a time delay between a prompt gamma associated with emission of the positron and the detection of the photons;
for each of multiple voxels of the subject of the PET scanning, estimating a spatial distribution of the annihilated positrons and lifetimes of the positrons or of positronia formed from the positrons; and
generate and display the positron lifetime image of the subject.

13. The system of claim 12, wherein the memory further stores instructions that, when executed, cause the one or more processors to:
construct a total activity image based on the detected photons;
construct one or more estimated intensity-weighted moment images based on the quantity of coincidence events detected in each of multiple LORs; and
generate the positron lifetime image by determining, for each of the multiple voxels, the ratio of the intensity-weighted moment images over the activity image and calculating the lifetime image from the moment images.

14. The system of claim 13, wherein:
generating the activity image comprises, for each coincidence event, correlating the detected photons to a voxel from which the photons originated.

15. The system of claim 13, further comprising:
modeling the time delays of coincidence events as a summation of exponential decays convolved by a Gaussian function characterized by a time resolution of the PET scanner.

16. The system of claim 12, wherein the memory further stores instructions that, when executed, cause the one or more processors to:
construct a total activity image based on the detected photons;
construct a lifetime-weighted image based on the coincidence events detected in each of multiple LORs; and
construct the positron lifetime image by determining, for each of the multiple voxels, the ratio of the lifetime-weighted image to the total activity image.

17. The system of claim 16, wherein the memory further stores instructions that, when executed, cause the one or more processors to:
aggregate the time delays of coincidence events in each of multiple lines of response (LOR);
weight the coincidence events by their time delays; and
sum the weights of the coincidence events.

18. The system of claim 12, wherein the memory further stores instructions that, when executed, cause the one or more processors to:
sort the coincidence events into multiple time intervals based on the time delays;
for each of the multiple time intervals, use the coincidence events occurring during the window to generate an intermediate image associated with the time window; and
fit a curve to the multiple intermediate images to produce, for each of multiple voxels within the subject, a corresponding portion of the positron lifetime image.

19. The system of claim 18, wherein the memory further stores instructions that, when executed, cause the one or more processors to:
iterate between updating the intermediate image using the estimated lifetimes and fitting a curve to the intermediate images to obtain the positron lifetime image.

20. The system of claim 18, wherein each coincidence event further comprises:
detection times of two annihilation photons caused by annihilation one or more positrons; and
a detection time of a prompt gamma.

21. A non-transitory computer-readable medium storing instructions that, when executed by a processor, cause the processor to perform a method of generating a positron lifetime image, the method comprising:
operating a Positron Emission Tomography (PET) scanner to collect coincidence events involving a subject of the scanning, wherein each coincidence event involves (a) a line of response (LOR) in which photons caused by annihilation of a positron are detected and (b) a time delay between a prompt gamma associated with emission of the positron and the detection of the photons;
for each of multiple voxels of the subject of the PET scanning, estimating a spatial distribution of the annihilated positrons and lifetimes of the positrons or of positronia formed from the positrons; and
generating and displaying the positron lifetime image of the subject.
